SEQUENCE LISTING (6 sequences)

SEQ ID NO: 1 - DNA, 37 bp, Artificial Sequence
  synthetic primer
cgacttttgctcatattattagccaagttccaggacc

SEQ ID NO: 2 - DNA, 37 bp, Artificial Sequence
  synthetic primer
ggtcctggaacttggctaataatatgagcaaaagtcg

SEQ ID NO: 3 - DNA, 37 bp, Artificial Sequence
  synthetic primer
cgacttttgctcatattatgagccaagttccaggacc

SEQ ID NO: 4 - DNA, 37 bp, Artificial Sequence
  synthetic primer
ggtcctggaacttggctcataatatgagcaaaagtcg

SEQ ID NO: 5 - DNA, 44 bp, Artificial Sequence
  synthetic primer
gaagatcctcttctcttcctctacatcaaatttgtcagctattg

SEQ ID NO: 6 - DNA, 44 bp, Artificial Sequence
  synthetic primer
caatagctgacaaatttgatgtagaggaagagaagaggatcttc

(19) United States
(12) Patent Application Publication
(10) Pub. No.: US 2010/0041110 A1
(43) Pub. Date: Feb. 18, 2010

(54) R-Hydroxynitrile Lyases Having Improved Substrate Acceptance and the Use Thereof

(76) Inventors: Roland Weis, Graz (AT); Anton Glieder, Gleisdorf (AT); Karl Gruber, Graz (AT); Wolfgang Skrang, Wien (AT); Oliver Maurer, Kirchberg/Thening (AT); Richard Gaisberger, Graz (AT)

Correspondence Address: NIXON & VANDERHYE, PC, 901 NORTH GLEBE ROAD, 11TH FLOOR, ARLINGTON, VA, 22203, US

(21) Appl. No.: 11/795,685

(22) PCT Filed: Dec. 28, 2005
(86) PCT No.: PCT/EP2005/014080

§ 371 Date: Jul. 20, 2007

(30) Foreign Application Priority Data
Jan. 20, 2005 (AT) A 84/2005

(51) Int. Cl. C12P 13/00; C12N 9/88

(52) U.S. Cl. 435/128; 435/232

(57) ABSTRACT

R-hydroxynitrile lyases having an improved substrate acceptance, increased activity and increased selectivity, in which there is replacement in the amino acid sequence of R-hydroxynitrile lyases from the Rosaceae family either a) of the amino acid residue which corresponds to position 360 of the mature PaHNL5 protein by another apolar amino acid or a neutral amino acid and/or b) of the amino acid residue which corresponds to position 225 of the mature PaHNL5 protein by another polar amino acid, it also being possible where appropriate for 1 to 20 further residues in the active center or in the hydrophobic channel leading to the active center to be replaced.

Biocatalytic processes have become very important for the chemical industry. The carrying-out of chemical reactions with the assistance of biological catalysts is in this connection of interest especially in areas of application in which it is possible to exploit the property of enzymes, which is often present, of preferentially converting or forming one of the two enantiomers in chemical reactions with chiral or prochiral components.

Essential preconditions for exploiting these favorable properties of enzymes are their availability in the quantities required industrially and a sufficiently high reactivity, as well as stability under the actual conditions of an industrial process.

A particularly interesting class of chiral chemical compounds are cyanohydrins. Cyanohydrins are important for example in the synthesis of α-hydroxy acids, α-hydroxy ketones, β-amino alcohols, which are used for obtaining biologically active substances, e.g. active pharmaceutical ingredients, vitamins or pyrethroid compounds.

These cyanohydrins are prepared by addition of hydrocyanic acid onto the carbonyl group of a ketone or aldehyde.

It has been possible to achieve the industrial preparation of chiral compounds such as, for example, (S)-cyanohydrins by making the enzyme (S)-hydroxynitrile lyase from Hevea brasiliensis available, as described for example in WO 97/03204, EP 0 951561 and EP 0 927 766.

However, there is a multiplicity of interesting chemical compounds for which the R enantiomers are important for industrial applications. To date, only processes for preparing a number of products which can be employed only on the laboratory scale have been described (e.g.: EP 0 276 375, EP 0 326 063, EP 0 547 655). The enzyme preparations employed in these cases were mainly those obtained from plants of the Rosaceae family, for example from almond kernels (Prunus amygdalus).

Further R-HNLs which have been employed to date are, for example, those from linseed seedlings (Linum usitatissimum; LuHNL) which were cloned as first gene of an R-HNL and were expressed in E. coli and Pichia pastoris, or R-HNL from Phlebodium aureum.

Advantageous reaction parameters described in the literature for obtaining products with high optical purity are low temperatures (e.g. Persson et al.; Enzyme and Microbial Technology 30(7), 916-923; 2002), a pH below 4 (e.g. Kragl et al.; Annals of the New York Academy of Science; 613 (enzyme Eng. 10), 167-75, 1990), and the use of 2-phase systems (for example EP 0 547 655) or of emulsions (e.g. EP 1 238 094).

Unfortunately, most R-HNLs have half-lives of less than one hour at a pH below 4. EP 1223220 A1 describes recombinant enzymes which are prepared by cloning a gene from Prunus amygdalus, which codes for an R-HNL isoenzyme, for example for isoenzyme 5 (PaHNL5), and by heterologous expression for example in Pichia pastoris, which are distinguished, as is evident from the examples, by a considerably increased stability at low pH values compared with the other known R-HNLs.

A disadvantage which has been found is that the substrate acceptance is unsatisfactory, because conversion of some substrates in the presence of, for example, recombinant PaHNL5 takes place at a distinctly lower reaction rate than in the presence of commercially available vegetable, native (R)-HNL preparations from almond kernels.

WO 2004/083424 describes mutants of these recombinant HNLs in which a residue from the group of alanine, phenylalanine, leucine or isoleucine in the active center is replaced by other residues, resulting in an increase in the substrate acceptance in particular for substituted benzaldehydes. One example is A111G mutant.

However, since there is still a great need in this area for enzymes which firstly can be provided on a sufficient industrial scale and cost-effectively for technical conversions and which have an improved substrate acceptance and thus increased activity, increased selectivity, and an increased stability, it was an object of the invention to find novel mutants of R-hydroxynitrile lyases from the Rosaceae family which satisfy these requirements.

It has unexpectedly been possible to achieve this object by specific mutations of R-hydroxynitrile lyases from the Rosaceae family, such as, for example, the PaHNL5 from EP 1223220 A1.

The invention accordingly relates to R-hydroxynitrile lyases having an improved substrate acceptance, increased activity and increased selectivity, which are characterized in that

there is replacement in the amino acid sequence of R-hydroxynitrile lyase from the Rosaceae family
either
a) of the amino acid residue which corresponds to position 360 of the mature PaHNL5 protein by another apolar amino acid or a neutral amino acid and/or
b) of the amino acid residue which corresponds to position 225 of the mature PaHNL5 protein by another polar amino acid,
   it also being possible where appropriate for 1 to 20 further residues in the active center or in the hydrophobic channel leading to the active center to be replaced.

The R-HNLs of the invention are mutants of R-hydroxynitrile lyase from the Rosaceae family.

It is possible to employ as initial basis for preparing the mutants of the invention native R-HNLs from the Rosaceae family, such as, for example, R-HNLs from Prunus amygdalus (PaHNL), Prunus serotina (PsHNL), Prunus laurocerasus, Prunus lyonii, Prunus armeniaca, Prunus persica, Prunus domestica (PdHNL), Malus communis, etc. or recombinant R-HNLs, as disclosed for example in EP 1223220.

The native R-HNLs which are preferably employed are R-HNLs from Prunus amygdalus (PaHNL), Prunus domestics (PdHNL) or from Prunus serotina (PsHNL).

Preferred recombinant R-HNLs are recombinant R-HNLs from Prunus domestica (PdHNL), in particular PdHNL1, and the recombinant R-HNLs PaHNL1 to PaHNL5 which are described in EP 1223220, with particular preference for recombinant PaHNL5.

The R-HNLs to be modified may moreover be in the form of an altered sequence which is obtained for example by exchange of the first amino acid(s) in the sequence or by deletion of the first amino acid(s) or by attachment of further amino acids, such as, for example, GluAlaGluAla, or by fusion with other isoenzymes. For example PaHNL5 can be fused to PaHNL4.

A further possibility before the mutation in the active center is to exchange the natural or vegetable signal sequence for another signal sequence such as, for example, for the signal sequence of the alpha mating factor from Saccharomyces cerevisiae (alpha-MF), Saccharomyces cerevisiae invertase (SUC2), Pichia killer toxin signal sequence, α-amylase, Pichia pastoris acid phosphatase (PHO1), Phaseolus vulgaris agglutinin (PHA-E); glycoamylase signal sequence from Aspergillus niger (glaA), glucose oxidase (GOX) signal sequence from Aspergillus niger, Sec10 signal sequence from Pichia pastoris, signal sequence of the 28 kD subunit of the killer toxin from Kluyveromyces lactis, BSA signal sequence, etc., or a recombinant signal sequence thereof. The signal sequences may moreover comprise point mutations. Suitable signal sequences and their mutants are described for example in Heijne G. et al., FEBS Letters 244 (2), 439-46 (1989), EP 19911213, Paifer et al., Biotecnologia Aplicada 10(1), 41-46, (1993), Raemaekers et al., European Journal of Biochemistry 265(1), 394-403 (1999) etc.

The vegetable signal sequence is preferably replaced by the signal sequence of the alpha mating factor from Saccharomyces cerevisiae.

The R-HNLs of the invention are prepared by site-specific mutagenesis, for example using the QuikChange (XL) Site Directed Mutagenesis Kit, QuikChange Multi Site Directed Mutagenesis Kit (from Stratagene), and kits from Invitrogen (e.g. GeneTailor Site-Directed Mutagenesis Kit), Clontach (e.g. Site-Directed Mutagenesis Transformer Kit) or Promega etc. in accordance with the manufacturer's instructions or by other conventional methods as described for example in Current Protocols in Molecular Biology, Ausubel et al., 2004.

Site-directed mutagenesis kits are systems ready for use for preparing specific mutants and are sold commercially for example by Stratagene Cloning Systems, La Jolla, Calif. (USA).

In the site-specific mutagenesis, there is according to the invention replacement either
a) of the amino acid residue which corresponds to position 360 of the mature PaHNL5 protein by another apolar amino acid or a neutral amino acid and/or
b) of the amino acid residue which corresponds to position 225 of the mature PaHNL5 protein by another polar amino acid.

A valine residue is present at position 360 of the mature PaHNL5 protein, and an asparagine residue is present at position 225. The residues corresponding to this position in other R-HNLs can easily be determined by a multiple alignment. FIG. 1 depicts such a multiple alignment for various known HNL sequences of the Rosaceae family. The sequences are in this case depicted without signal sequences.

The valine residue or the corresponding amino acid at this position is thus replaced according to the invention by another apolar amino acid such as, for example, isoleucine, methionine, alanine, phenylalanine or leucine, or by a neutral amino acid such as, for example, glycine or tryptophan. Replacement by leucine, isoleucine or methionine is preferred.

The asparagine residue or the corresponding amino acid at this position is replaced according to the invention by another polar amino acid such as, for example, serine, cysteine, lysine, histidine, glutamic acid, glutamine or aspartic acid. Replacement by serine or aspartic acid is preferred.

The mutants of the invention may also where appropriate have 1 to 20, preferably up to 15, further mutations such as, for example, mutations in the active center, for example the mutation A111G disclosed in WO 2004/083424, or have for example the mutation L331A where appropriate in the hydrophobic channel leading to the active center.

The active center may in this connection be defined as the approximately 10-12 Angstömm spherical space around the substrate-binding site.

The numberings are derived from the corresponding positions in the mature unmodified recombinant R-hydroxynitrile lyase PaHNL5, but the positions can be shifted according to the abovementioned modifications of the sequence, such as, for example, fusion, random insertions or deletions, truncation or extension of the sequence.

(Heterologous or secretory) expression then takes place, preferably secretory expression in suitable microorganisms such as, for example, in Pichia pastoris, Saccharomyces cerevisiae or Escherichia coli, Bacillus subtilis, Pseudomonas fluorescens, Kluyveromyces lactis, Aspergillus niger, Penicillium chrysogenum, Pichia methanolica, Pichia polymorpha, Hansenula polymorpha, Pichia anomala, Schizosaccharomyces pombe, etc.

The resulting R-HNL mutants of the invention are purified by standard methods, for example in analogy to Dreveny et al.; Structure (Cambridge; MA, United States) 9(9), 803-815; 2001.

The R-HNL mutants of the invention are suitable for the preparation of enantiopure cyanohydrins in a conversion rate, activity and selectivity which are increased compared with the prior art.

The invention accordingly relates further to the use of the R-HNL mutants of the invention for preparing enantiopure cyanohydrins.

The R-HNL mutants of the invention are employed in particular with aliphatic and aromatic aldehydes and ketones as substrates.

Aldehydes mean in this connection aliphatic, aromatic or heteroaromatic aldehydes. Aliphatic aldehydes mean in this connection saturated or unsaturated, aliphatic, straight-chain, branched or cyclic aldehydes. Preferred aliphatic aldehydes are straight-chain or branched aldehydes having in particular 2 to 30 C atoms, preferably from 4 to 18 C atoms, which are saturated or mono- or polyunsaturated. The aldehyde may in this connection have both C—C double bonds and C—C triple bonds. The aliphatic, aromatic or heteroaromatic aldehydes may moreover be unsubstituted or substituted by groups which are inert under the reaction conditions, for example by optionally substituted aryl or heteroaryl groups, such as phenyl, phenoxy or indolyl groups, by halogen, hydroxy, hydroxy-C₁-C₅-alkyl, C₁-C₅-alkoxy, C₁-C₅-alkylthio, ether, alcohol, carboxylic ester, nitro or azido groups.

Examples of preferred aliphatic aldehydes are butanal, 2-butenal, 3-phenylpropanal, 3-phenylpropenal, 3-phenylpropynal, pivalaldehyde, hydroxypivalaldehyde, etc. Examples of aromatic or heteroaromatic aldehydes are benzaldehyde and variously substituted benzaldehydes such as, for example, 2-chlorobenzaldehyde, 3-chlorobenzaldehyde, 4-chlorobenzaldehyde, 3,4-difluorobenzaldehyde, 3-phenoxybenzaldehyde, 4-fluoro-3-phenoxybenzaldehyde, hydroxybenzaldehydes, methoxybenzaldehydes, also furfural, methylfurfural, anthracene-9-carbaldehyde, furan-3-carbaldehyde, indole-3-carbaldehyde, naphthalene-1-carbaldehyde, phthalaldehyde, pyrazole-3-carbaldehyde, pyrrole-2-carbaldehyde, thiophene-2-carbaldehyde, isophthalaldehyde or pyridinealdehydes, thienylaldehydes, etc.

Ketones are aliphatic, aromatic or heteroaromatic ketones in which the carbonyl-carbon atom has different substituents. Aliphatic ketones mean saturated or unsaturated, straight-chain, branched or cyclic ketones. The ketones may be saturated or mono- or polyunsaturated. They may be unsubstituted or substituted by groups which are inert under the reaction conditions, for example by optionally substituted aryl or heteroaryl groups such as phenyl or indolyl groups, by halogen, ether, alcohol, carboxylic ester, nitro or azido groups.

Examples of aromatic or heteroaromatic ketones are acetophenone, indolylacetone, etc.

Aldehydes and ketones suitable according to the invention are known or can be prepared in a conventional way.

The substrates are converted in the presence of the HNLs of the invention with a cyanide group donor.

Suitable as cyanide group donor is hydrocyanic acid, alkali metal cyanides or a cyanohydrin of the general formula I

R₁R₂C(OH)(CN).

In formula I, R₁ and R₂ are independently of one another hydrogen or an unsubstituted hydrocarbon group, or R₁ and R₂ together are an alkylene group having 4 or 5 C atoms, with R₁ and R₂ not both being hydrogen. The hydrocarbon groups are aliphatic or aromatic, preferably aliphatic, groups. R₁ and R₂ are preferably alkyl groups having 1-6 C atoms, and the cyanide group donor is very preferably acetone cyanohydrin.

The cyanide group donor can be prepared by known processes. Cyanohydrins, especially acetone cyanohydrin, can also be purchased.

The cyanide group donor employed is preferably hydrocyanic acid (HCN), KCN, NaCN, or acetone cyanohydrin, particularly preferably hydrocyanic acid.

The hydrocyanic acid can moreover be liberated only shortly before the reaction from one of its salts such as, for example, NaCN or KCN and be added undiluted or in dissolved form to the reaction mixture.

The conversion can be carried out in an organic, aqueous or 2-phase system or in emulsion, and without diluent.

An aqueous solution or buffer solution comprising the HNL of the invention is used as aqueous system. Examples thereof are Na citrate buffer, phosphate buffer, etc.

It is possible to use as organic diluent, water-immiscible or slightly water-miscible aliphatic or aromatic hydrocarbons, which are optionally halogenated, alcohols, ethers or esters or mixtures thereof or the substrate itself. Toluene, xylenes, methyl tert-butyl ether (MTBE), diisopropyl ether, dibutyl ether and ethyl acetate or mixtures thereof are preferably employed.

The HNLs of the invention can moreover be present either as such or immobilized, for example on a carrier or as a “cross-linked enzyme aggregate” in the organic diluent, but the conversion can also take place in a two-phase system or in an emulsion with nonimmobilized HNL.

The conversion moreover takes place at temperatures of from −10° C. to +50° C., preferably at −5° C. to +45° C.

The pH of the reaction mixture can be from 1.8 to 7, preferably from 2 to 5 and particularly preferably from 2.5 to 3.5.

### EXAMPLE 1

### Site-Specific Mutagenesis

In each case 10 ng of the expression plasmids pHILDPaHNL5α_L1Q (PaHNL5 with alpha factor signal sequence, described in WO 2004/083424 and Angew. Chem. Int. Ed. Engl. 2003; 42, 4815) (mutants V360I, V360M and N225S) and pHILDPaHNL5α_L1Q, A111G (WO 2004/083424, Angew. Chem. Int. Ed. Engl. 2003; 42, 4815) (mutant A111GV360I) were employed as template for the mutagenesis reaction using the QuikChange XL Site Directed Mutagenesis Kit from Stratagene (Cat. #200516). 200 ng of each of the two mutagenesis primers were employed for the reaction. The following temperature program was used:

A) denaturation at 95° C. for one minute
B) 18 cycles with 50 sec at 95° C., 50 sec at 60° C. and 20 min at 68° C.
C) extension for 7 min at 68° C.

The template DNA was digested off with DpnI, as described in the kit protocol, and 2 μl of the mixture were employed as described for transforming ultracompetent E. coli XL10 Gold cells. Plasmid DNA was prepared from the transformants and sequenced. Plasmids from mutants having the correct sequence in the region of the coding DNA insert were replicated and transformed into Pichia pastoris GS115 with the aid of the standard Invitrogen procedure.

Several histidine-autotrophic Pichia transformants were cultivated in deep well plates, and the activity of the culture supernatants was determined with racemic mandelonitrile in 96-well plates. Clones having in each case the highest enzymic activity of the individual mutants were selected for shaken flask experiments. The enzymic activity of the culture supernatants was determined using the substrate mandelonitrile.

### PCR Primers for the Site-Specific Mutagenesis:

For mutation V360I:
V360Iforw:
5′-cgacttttgctcatattattagccaagttccaggacc-3′
V360Irev:
5′-ggtcctggaacttggctaataatatgagcaaaagtcg-3′
For mutation V360M:
V360Mforw:
5′-cgacttttgctcatattatgagccaagttccaggacc-3′
V360Mrev:
5′-ggtcctggaacttggctcataatatgagcaaaagtcg-3′
For mutation N225S:
N225Sf:
5′-gaagatcctcttctcttcctctacatcaaatttgtcagctattg-3′
N225Sr:
5′-caatagctgacaaatttgatgtagaggaagagaagaggatcttc-3′

### EXAMPLE 2

### Purification and Characterization of the Enzyme Variants

The specific activity of the respective mutants with different substrates was determined by carrying out several shaken flask cultures with each of the expression clones. The culture supernatant was concentrated by ultrafiltration (30 kDa cutoff) using 20 ml Vivaspin PES centrifugation columns from Sartorius (Gottingen, D) and then purified by chromatography.

Before the purification, the concentrated culture supernatant was equilibrated with the low-salt binding buffer A by repeated dilution and concentration with binding buffer A (20 mM citrate-phosphate buffer, pH 5.5) in 30 kDa ultrafiltration centrifugation modules (Vivaspin, Sartorius), and then purified on a Q-Sepharose Fast Flow (QFF) anion exchange column with a column volume of 10 ml in an ÄKTApurifier 10 FPLC system from Amersham Biosciences UK Limited (Buckinghamshire, GB). Elution took place with elution buffer B (20 mM citrate-phosphate buffer+1M NaCl, pH 5.5), using the following gradient profile for the different variants of PaHNL5 from heterologous production with Pichia pastoris:

one column volume as washing step proved to be ideal for washing out all unbound protein constituents. The concentration of buffer B (elution buffer: 20 mM citrate-phosphate buffer, 1M NaCl, pH 5.5) was raised in half a column volume to 4% and subsequently increased to 48% in a further column volume. The next step was to increase the concentration of elution buffer B to 70%, using 1½ column volumes in this case.

Finally, the concentration was raised to the maximum of 100% in one column volume and was in conclusion left thereat for a further column volume (washing step without fractionation).

Those fractions which ought, according to evaluation of the chromatogram, to contain protein (depending on the peak position) underwent determination of the protein content using the Biorad (Hercules, Calif.) protein assay (Bradford method) and of the enzymic activity using the substrate mandelonitrile. The 2-3 fractions with the highest activity were pooled and employed for analyzing the enzyme characteristics. The protein concentration was carried out with a Biorad (Hercules, Calif.) protein assay (Bradford). The standard used for producing a calibration line was native PaHNL from Sigma (M-6782 Lot 41H4016). The culture supernatants were concentrated ˜20-fold by cross-flow filtration and then purified by chromatography. Samples were taken of the purified enzymes and loaded directly onto a gel (protein gel NuPAGE 4-12% bis gel 1 mm×17 well; Invitrogen), or ˜500 ng were deglycosylated with endoglycosidase H (#P0702L, NEB) (according to the procedure supplied) and then loaded. The standard used was “SeeBlue Plus2 Pre-Stained Standard” from Invitrogen (Carlsbad, USA).

To compare the substrate specificities, the protein concentration of the purified enzymes and the protein content in the culture supernatant were measured using the Biorad protein assay (Hercules, Calif.), and the specific activities were compared with 3-phenylpropionaldehyde and 3-phenylpropenaldehyde by GC:

for this purpose, 15 mmol of substrate were dissolved in 2.1 ml of tert-butyl methyl ether (MTBE). Various amounts of the appropriate PaHNL were diluted with 50 mM K₂HPO₄/citrate buffer of pH 3.4 to a final volume of 3.6 ml, the buffer was again adjusted to pH 3.4 and then mixed with the substrate in MTBE in 20 ml glass vials. The solution was cooled to 10° C., and 1.2 ml of HCN was added with a syringe and stirred at 700 rpm and 10° C. on a magnetic stirrer. Samples were taken at various times, derivatized with acetic anhydride in the presence of pyridine and dichloromethane, and analyzed by GC on a cyclodextrin column (CP-Chirasil-Dex CB) or by HPLC.

TABLE 1
Conversion of 15 mmol of 3-phenylpropionaldehyde
with PaHNL5-L1Q
(WO 2004/083424) and mutants of the invention

| | | Reaction time | | | | | |
|---|---|---|---|---|---|---|---|
| | | 1 h | | 2 h | | 4 h | |
| | | conv | | conv | | conv | |
| Entry | Enzyme [1 mg] | (%) | ee (%) | (%) | ee (%) | (%) | ee (%) |
| 1 | PaHNL5-L1Q | 72.0 | 89.0 | 86.7 | 90.1 | 95.8 | 90.2 |
| 2 | A111GV360I | 70.1 | 90 | 83.3 | 90.6 | 94.0 | 91.8 |
| 3 | N225S | n.d | n.d | 93.0 | 93.3 | n.d | n.d |
| 4 | V360M | 78.0 | 93.6 | 92.5 | 94.0 | 97.2 | 94.6 |
| 5 | V360I | 85.7 | 96.0 | 95.9 | 96.6 | 98.0 | 96.7 |

n.d: not determined

TABLE 2
Specific activity and TOF (turnover frequency) values of PaHNL5-L1Q
and mutants of the invention for the substrate 3-phenylpropionaldehyde

| | Specific activity [μmol min⁻¹ mg] | TOF [s⁻¹] |
|---|---|---|
| PaHNL5-L1Q | 2588 ± 215 | 2497 ± 208 |
| A111GV360I | 3501 ± 215 | 3379 ± 208 |
| V360M | 7059 ± 867 | 6812 ± 837 |
| V360I | 14918 ± 431 | 14397 ± 416 |

TABLE 3
Conversion of 15 mmol of 3-phenylpropenaldehyde with
PaHNL5-L1Q and mutants of the invention

| | | 2 h | | 3 h | |
|---|---|---|---|---|---|
| Entry | Enzyme [0.4 mg] | conv (%) | ee (%) | conv (%) | ee (%) |
| 1 | PaHNL5-L1Q | 22 | 96.2 | 30 | 96.4 |
| 2 | A111GV360I | 36 | 96.8 | 47 | 96.8 |
| 4 | V360M | 14 | 92.7 | 18 | 92.6 |
| 5 | V360I | 90 | 97.9 | 97 | 97.6 |

TABLE 4
Specific activity and TOF values of PaHNL5-L1Q and mutants of the
invention for the substrate 3-phenylpropenaldehyde

| | Specific activity [μmol min⁻¹ mg] | TOF [s⁻¹] |
|---|---|---|
| PaHNL5-L1Q | 114 ± 20 | 110 ± 19 |
| A111GV360I | 154 ± 17 | 149 ± 16 |
| V360I | 475 ± 33 | 458 ± 32 |

### EXAMPLE 3

### Determination of the Activity of PaHNL5-L1Q, N225S in the Cleavage of Mandelonitrile

Cultivation of P. pastoris GS115 PaHNL5-L1Q, N225S (as described above) and harvesting of the culture supernatant by centrifugation were followed by approximately 20-fold concentration of the latter by ultrafiltration (30 kDa cutoff). Determination of the protein concentration via the Bradford method and determination of the activity in the mandelonitrile cleavage reaction using the method described above afforded the value for the specific activity in U/mg of enzyme. Repetition of the complete procedure three times made it possible to state an average and a standard deviation for the specific activity of PaHNL5-L1Q, N225S. This was 525+/−30 U/mg which is about 1.6 times that of the recombinant wild-type enzyme PaHNL5-L1Q.

1. A R-hydroxynitrile lyase having an improved substrate acceptance, increased activity and increased selectivity, wherein there is replacement in the amino acid sequence of a R-hydroxynitrile lyase from the Rosaceae family
either
a) of the amino acid residue which corresponds to position 360 of the mature PaHNL5 protein by another apolar amino acid or a neutral amino acid and/or
b) of the amino acid residue which corresponds to position 225 of the mature PaHNL5 protein by another polar amino acid,
it also being possible where appropriate for 1 to 20 further residues in the active center or in the hydrophobic channel leading to the active center to be replaced.

2. The R-hydroxynitrile lyase according to claim 1, wherein the replacement is carried out in a R-hydroxynitrile lyase selected from the group consisting of Prunus amygdalus, Prunus serotina, Prunus laurocerasus, Prunus lyonii, Prunus armeniaca, Prunus persica, Prunus domestica, Malus communis, and in recombinant R-hydroxynitrile lyases thereof.

3. The R-hydroxynitrile lyase according to claim 1, wherein—the R-hydroxynitrile lyase to be modified is in the form of the complete sequence or of the sequence modified by a replacement of the first amino acid(s), random insertions or deletions, or of the sequence truncated by deletion of the first amino acid(s) or the sequence extended by attaching further amino acids or by fusion.

4. The R-hydroxynitrile lyase according to claim 1, wherein before the mutation the natural or vegetable signal sequence is exchanged for the signal sequence of the alpha mating factor from Saccharomyces cerevisiae, Saccharomyces cerevisiae invertase, Pichia killer toxin signal sequence, α-amylase, Pichia pastoris acid phosphatase, or Phaseolus vulgaris agglutinin; glycoamylase signal sequence from Aspergillus niger, glucose oxidase signal sequence from Aspergillus niger, Sec10 signal sequence from Pichia pastoris, signal sequence of the 28 kD subunit of the killer toxin from Kluyveromyces lactis or the BSA signal sequence, or by a recombinant signal sequence thereof, or by one of the abovementioned signal sequences with point mutation.

5. The R-hydroxynitrile lyase according to claim 1, wherein preparation takes place by site-specific mutagenesis with subsequent heterologous or secretory expression in a microorganism selected from the group consisting of Pichia pastoris, Saccharomyces cerevisiae, Escherichia coli, Bacillus subtilis, Pseudomonas fluorescens, Kluyveromyces lactis, Aspergillus niger, Penicillium chrysogenum, Pichia methanolica, Pichia polymorpha, Hansenula polymorpha, Pichia anomala, and Schizosaccharomyces pombe.

6. The R-hydroxynitrile lyase according to claim 1, wherein the residue which corresponds to position 360 of the mature PaHNL5 protein is replaced by an apolar amino acid selected from the group consisting of isoleucine, methionine, alanine, phenylalanine and leucine, or by a neutral amino acid selected from the group consisting glycine and tryptophan.

7. The R-hydroxynitrile lyase according to claim 1, wherein the residue which corresponds to position 225 of the mature PaHNL5 protein is replaced by a polar amino acid selected from the group consisting of serine, cysteine, lysine, histidine, glutamic acid, glutamine and aspartic acid.

8. Use of a R-hydroxynitrile lyase according to claim 1 for preparing enantiopure cyanohydrins.

9. Process for preparing enantiopure cyanohydrins, wherein the process comprises: converting aliphatic, aromatic or heteroaromatic aldehydes or ketones in the presence of a cyanide group donor with a R-hydroxynitrile lyase according to claim 1 in an organic, aqueous or two-phase system or in an emulsion or without diluent at a temperature of from −10° C. to +50° C. and at a pH of from 1.8 to 7.
